(19) United States
(12) Patent Application Publication
(10) Pub. No.: US 2003/0036081 A1
(43) Pub. Date: Feb. 20, 2003

(54) Distributed system for epigenetic based prediction of complex phenotypes

(75) Inventors: Peter Adorjan, Berlin (DE); Alexander Olek, Berlin (DE); Christian Piepenbrock, Berlin (DE)

Correspondence Address: DAVIDSON, DAVIDSON & KAPPEL, LLC, 485 SEVENTH AVENUE, 14TH FLOOR, NEW YORK, NY, 10018, US

(73) Assignee: Epigenomics AG, Berlin (DE)

(21) Appl. No.: 10/186,545

(22) Filed: Jul. 1, 2002

Related U.S. Application Data
(60) Provisional application No. 60/302,490, filed on Jul. 2, 2001.

(51) Int. Cl. C12Q 1/68

(52) U.S. Cl. 435/6

(57) ABSTRACT

This invention concerns systems for collecting and storing epigenetic and phenotypic information about samples in order to measure and analyse tissue samples and/or cell lines, where the epigenetic parameter is DNA methylation and the phenotypic parameters describe an individual. The method includes parameters such as the diagnosis of diseases and/or drug resistance, wherein the correlation of the epigenetic with the phenotypic parameters is done substantially without human intervention.

This application claims priority from U.S. Provisional Application Serial No. 60/302,490 filed Jul. 2, 2001, the disclosure of which is hereby incorporated by reference.

### BACKGROUND OF THE INVENTION

1. Field of the Invention

This invention concerns systems for collecting and storing epigenetic and phenotypic information about samples in order to measure and analyse tissue samples and/or cell lines, where the epigenetic parameter is DNA methylation and the phenotypic parameters describe an individual. The method includes parameters such as the diagnosis of diseases and/or drug resistance, wherein the correlation of the epigenetic with the phenotypic parameters is done substantially without human intervention.

2. Description of Related Art

This part of the disclosure shall describe the general background of the invention and is not meant to be limiting for the invention in any way. All cited references are included into the description by reference in their entirety. The levels of observation that have been well studied by the methodological developments of recent years in molecular biology, are the genes themselves, the translation of these genes into RNA, and the resulting proteins. The question of which gene is switched on at which point in the course of the development of an individual, and how the activation and inhibition of specific genes in specific cells and tissues are controlled is correlatable to the degree and character of the methylation of the genes or of the genome. In this respect, pathogenic conditions may manifest themselves in a changed methylation pattern of individual genes or of the genome.

Molecular portraits, such as mRNA expression or DNA methylation patterns, have been shown to be strongly correlated with phenotypical parameters. These molecular patterns can be revealed routinely on a genomic scale. However, class prediction based on these patterns is an under-determined problem, due to the extreme high dimensionality of the data compared to the usually small number of available samples. This makes a reduction of the data dimensionality necessary. By comparing several feature selection methods, the right dimension reduction strategy is of crucial importance for the classification performance.

In recent years there has been a large interest in the analysis of mRNA expression by using microarrays (Lockhart, D. J., Winzeler, E. A., “Genomics, gene expression and DNA arrays.” Nature 405:827-836 (2000). This technology makes it possible to look at thousands of genes, see how they are expressed as proteins and gain insight into cellular processes. An important and scientifically interesting application of this technology is the classification of tissue types (Golub, T. R., et al. “Molecular classification of cancer: Class discovery and class prediction by gene expression monitoring.” Science 286:531-537 (1999); Ben-Dor, A., et al. “Tissue classification with gene expression profiles.” RECOMB01, in press (2001); Weston J., et al. “Feature Selection for SVMs.” To appear in Advances in neural information processing systems 13. MIT Press, Cambridge, Mass. (2001)).

However, there are some practical problems with the large scale analysis of mRNA based microarrays. They are primarily impeded by the instability of mRNA (Emmert-Buck, T., et al. “Molecular profiling of clinical tissue specimens: feasibility and applications.” Am J Pathol. 156:1109-15 (2000). Also expression changes of only a minimum of a factor 2 can be routinely and reliably detected. Furthermore, sample preparation is complicated by the fact that expression changes occur within minutes following certain triggers. The inability to resolve the individual contributions of such influences on an expression profile, and difficulties with quantifying the gradual nature of the occurring changes complicates data analysis.

An alternative approach is to look directly at DNA methylation. Methylation is a modification of cytosine in the combination CpG that can occur either with or without a methyl group attached. The methylated CpG can be seen as a 5th base and is one of the major factors responsible for expression regulation (Robertson, K. D., Wolffe, A. P., “DNA methylation in health and disease.” Nature Reviews Genetics 1:11-19 (2000). Aberrant DNA methylation within CpG islands is common in human malignancies leading to abrogation or overexpression of a broad spectrum of genes. Abnormal methylation has also been shown to occur in CpG rich regulatory elements in intronic and coding parts of genes for certain tumours.

5-Methylcytosine is the most frequent covalent base modification in the DNA of eukaryotic cells. Therefore, the identification of 5-methylcytosine as a component of genetic information is of considerable interest. However, 5-methylcytosine positions cannot be identified by sequencing since 5-methylcytosine has the same base pairing behaviour as cytosine. Moreover, the epigenetic information carried by 5-methylcytosine is completely lost during PCR amplification.

A relatively new and currently the most frequently used method for analysing DNA for 5-methylcytosine is based upon the specific reaction of bisulfite with cytosine which, upon subsequent alkaline hydrolysis, is converted to uracil which corresponds to thymidine in its base pairing behaviour. However, 5-methylcytosine remains unmodified under these conditions. Consequently, the original DNA is converted in such a manner that methylcytosine, which originally could not be distinguished from cytosine by its hybridisation behaviour, can now be detected as the only remaining cytosine using “normal” molecular biological techniques, for example, by amplification and hybridisation or sequencing. All of these techniques are based on base pairing which can now be fully exploited. In terms of sensitivity, the prior art is defined by a method which encloses the DNA to be analysed in an agarose matrix, thus preventing the diffusion and renaturation of the DNA (bisulfite only reacts with single-stranded DNA), and which replaces all precipitation and purification steps with fast dialysis (Olek A, Oswald J, Walter J. A modified and improved method for bisulphite based cytosine methylation analysis. Nucleic Acids Res. Dec. 15, 1996; 24(24):5064-6). Using this method, it is possible to analyse individual cells, which illustrates the potential of the method. However, currently only individual regions of a length of up to approximately 3000 base pairs are analysed, a global analysis of cells for thousands of possible methylation events is not possible. However, this method cannot reliably analyse very small fragments from small sample quantities either. These are lost through the matrix in spite of the diffusion protection.

An overview of the further known methods of detecting 5-methylcytosine may be gathered from the following review article: Rein, T., DePamphilis, M. L., Zorbas, H., Nucleic Acids Res. 1998, 26, 2255.

To date, barring few exceptions (e.g., Zeschnigk M, Lich C, Buiting K, Doerfler W, Horsthemke B. A single-tube PCR test for the diagnosis of Angelman and Prader-Willi syndrome based on allelic methylation differences at the SNRPN locus. Eur J Hum Genet. March-April 1997; 5(2):94-8) the bisulfite technique is only used in research. Always, however, short, specific fragments of a known gene are amplified subsequent to a bisulfite treatment and either completely sequenced (Olek A, Walter J. The pre-implantation ontogeny of the H19 methylation imprint. Nat Genet. November 1997; 17(3):275-6) or individual cytosine positions are detected by a primer extension reaction (Gonzalgo M L, Jones P A. Rapid quantitation of methylation differences at specific sites using methylation-sensitive single nucleotide primer extension (MsSNuPE). Nucleic Acids Res. Jun. 15, 1997; 25(12):2529-31, WO 95/00669) or by enzymatic digestion (Xiong Z, Laird P W. COBRA: a sensitive and quantitative DNA methylation assay. Nucleic Acids Res. Jun. 15, 1997; 25(12):2532-4). In addition, detection by hybridisation has also been described (Olek et al., WO 99/28498).

Further publications dealing with the use of the bisulfite technique for methylation detection in individual genes are: Grigg G, Clark S. Sequencing 5-methylcytosine residues in genomic DNA. Bioessays. June 1994; 16(6):431-6, 431; Zeschnigk M, Schmitz B, Dittrich B, Buiting K, Horsthemke B, Doerfler W. Imprinted segments in the human genome: different DNA methylation patterns in the Prader-Willi/Angelman syndrome region as determined by the genomic sequencing method. Hum Mol Genet. March 1997; 6(3):387-95; Feil R, Charlton J, Bird A P, Walter J, Reik W. Methylation analysis on individual chromosomes: improved protocol for bisulphite genomic sequencing. Nucleic Acids Res. Feb. 25, 1994; 22(4):695-6; Martin V, Ribieras S, Song-Wang X, Rio M C, Dante R. Genomic sequencing indicates a correlation between DNA hypomethylation in the 5′ region of the pS2 gene and its expression in human breast cancer cell lines. Gene. May 19, 1995; 157(1-2):261-4; WO 97/46705, WO 95/15373 and WO 97/45560.

An overview of the Prior Art in oligomer array manufacturing can be gathered from a special edition of Nature Genetics (Nature Genetics Supplement, Volume 21, January 1999), published in January 1999, and from the literature cited therein.

Fluorescently labelled probes are often used for the scanning of immobilised DNA arrays. The simple attachment of Cy3 and Cy5 dyes to the 5′-OH of the specific probe are particularly suitable for fluorescence labels. The detection of the fluorescence of the hybridised probes may be carried out, for example via a confocal microscope. Cy3 and Cy5 dyes, besides many others, are commercially available.

Genomic DNA is obtained from DNA of cell, tissue or other test samples using standard methods. This standard methodology is found in references such as Fritsch and Maniatis eds., Molecular Cloning: A Laboratory Manual, 1989.

By the term “individual” is meant, for the purposes of the specification and claims to refer to any mammal, especially humans.

In the context of the present invention, “genetic parameters” are mutations and polymorphisms of genes associated with DNA adducts. To be designated as mutations are, in particular, insertions, deletions, point mutations, inversions and polymorphisms and, particularly preferred, SNPs (single nucleotide polymorphisms).

In the context of the present invention, “epigenetic parameters” are, in particular, cytosine methylations and further chemical modifications of DNA bases of genes associated with DNA adducts and sequences further required for their regulation. Further epigenetic parameters include, for example, the acetylation of histones which, however, cannot be directly analysed using the described method but which, in turn, correlate with the DNA methylation.

XML is a method for putting structured data in a text file. It is designed to enable the use of SGML (Standard Generalized Markup Language) on the World Wide Web. XML simplifies the levels of optionality in SGML, and allows the development of user-defined document types on the Web.

Using the XML terminology the transmission is defined in the DTD (Document type definition) files. DTD specifies the standard format of a mark-up language. So data are transmitted in a structured form, where the structure is defined by tags (or data fields identifiers) and the meaning (and type) of the tags is defined in DTD.

IPsec (Internet Protocol security) is designed to provide interoperable, high quality, cryptographically-based security. The set of security services offered includes access control, connectionless integrity, data origin authentication, protection against replays (a form of partial sequence integrity), confidentiality (encryption), and limited traffic flow confidentiality. These services are provided at the IP layer, offering protection for IP and/or upper layer protocols.

The Common Object Request Broker Architecture (CORBA), is the Object Management Group's answer to the need for interoperability among the rapidly proliferating number of hardware and software products available today. Simply stated, CORBA allows applications to communicate with one another no matter where they are located or who has designed them. CORBA 1.1 was introduced in 1991 by Object Management Group (OMG) and defined the Interface Definition Language (IDL) and the Application Programming Interfaces (API) that enable client/server object interaction within a specific implementation of an Object Request Broker (ORB). CORBA 2.0, adopted in December 1994, defines true interoperability by specifying how ORBs from different vendors can interoperate.

The (ORB) is the middleware that establishes the client-server relationships between objects. Using an ORB, a client can transparently invoke a method on a server object, which can be on the same machine or across a network. The ORB intercepts the call and is responsible for finding an object that can implement the request, pass it the parameters, invoke its method, and return the results. The client does not have to be aware of where the object is located, its programming language, its operating system, or any other system aspects that are not part of an object's interface. In so doing, the ORB provides interoperability between applications on different machines in heterogeneous distributed environments and seamlessly interconnects multiple object systems.

In fielding typical client/server applications, developers use their own design or a recognised standard to define the protocol to be used between the devices. Protocol definition depends on the implementation language, network transport and a dozen other factors. ORBs simplify this process. With an ORB, the protocol is defined through the application interfaces via a single implementation language-independent specification, the IDL. And ORBs provide flexibility. They let programmers choose the most appropriate operating system, execution environment and even programming language to use for each component of a system under construction. More importantly, they allow the integration of existing components. In an ORB-based solution, developers simply model the legacy component using the same IDL they use for creating new objects, then write “wrapper” code that translates between the standardised bus and the legacy interfaces.

CORBA is a single step on the road to object-oriented standardisation and interoperability. With CORBA, users gain access to information transparently, without them having to know what software or hardware platform it resides on or where it is located on an enterprises' network. The communications heart of object-oriented systems, CORBA brings true interoperability to today's computing environment.

### DESCRIPTION

No matter which biological platform technology or data-source will dominate the future health-care industry, there will by far be no product in such demand as tools for storage, administration, organisation, secure transfer and most important the interpretation of complex epigenetic data. In particular, when the focus of the sector turns from blueprint data to information on the epigenetics of individuals, an explosion of available data will result, unprecedented in industry.

With the advent of personalised medicines, literally gigabytes of data may be produced routinely in the diagnosis of every single individual with complex diseases. Also, as data storage and production will be increasingly distributed, retrieval and brokerage of data take place almost certainly via the internet. However, the development and introduction into clinical use of modem genetic systems is severely hampered by a lack of information technology infrastructure.

This invention, an epigenetic information method also comprises a method which consists of the following seven steps:

In the first step, tissue samples and cell lines are collected and stored in a systematic way. Along with these samples or cell lines, phenotypic information about these samples is collected, assigned to the samples and stored.

There are several ways how the collection of samples can be performed. Preferably, tissue samples are obtained from cell lines, biopsies, blood, sputum, stool, urine, cerebral-spinal fluid, tissue embedded in paraffin such as tissue from eyes, intestine, kidney, brain, heart, prostate, lung, breast or liver, histologic object slides, and all possible combinations thereof.

Clinical records are preferably entered in a systematic way into tables using computer interfaces. The data is anonymized and assigned to the samples. Then barcodes or other unique identifiers are attached.

The data generation process is completely integrated into a data and quality management system. Preferably, the genetic or epigenetic and phenotypic parameters are retrieved from data storage computer systems that are connected to the Internet or to a like distributed data exchange system. The progress of all processed samples is monitored. Massive amounts of both input (samples, clinical information) and output (molecular genetic) data are processed and handled.

Fresh tissue samples and cells are preferably stored at −80° C., tissue embedded in paraffin at room temperature, DNA in water is stored at −20° C. and TE buffered DNA at 4° C. The DNA is extracted according to standard protocols.

In the second step, a molecular biological system measures and analysis multiple genetic and/or epigenetic parameters from the tissues and cells. In a preferred embodiment, the epigenetic parameter is DNA methylation.

Preferably, the molecular biological system for ascertaining genetic and/or epigenetic parameters of tissues or cells comprises:

a. isolating a DNA-containing sample from an individual;

b. analysing cytosine methylation patterns and single nucleotide polymorphisms at selected sites of the DNA contained in the sample whereby a bisulphite treatment of the DNA is applied for the cytosine methylations;

c. providing data about the methylation status at selected sites of the DNA of the individual.

With respect to the entire epigenetic information method claimed, the phenotypic parameters are preferably on the cellular or molecular level. In a preferred embodiment, the cellular level consists of different types of tissues or deletions of a chromosome whereas the molecular level comprises the expression level of specific genes or the methylation status of selected CpGs.

In the third step, the collected are shipped in a systematic and standardised way to a central or multiple distributed database.

The phenotypic information about the patient preferably includes gender, age, diagnosis (with all diagnostic parameters like blood pressure, sugar level or stage of cancer), disease history (including the pathological information about the tissue sample of the patient like morphology, staging or grading), drug resistance, exposure to chemicals, life style and populational information. Cell lines are also connected with patient data and they have detailed pathological or morphological description.

Preferably, the collected phenotypic parameters from said input devices are sent to a server device over a communications network, wherein said server is located relatively remote from said input device.

In the fourth step, the measured genetic and epigenetic parameters are shipped in a systematic and standardised way to a central or multiple distributed database. This is backed by software to prepare raw data for interpretation by converting measured values, e.g. from scanners or mass spectrometry, into methylation information.

The data description and the data transfer is preferably done by using a standard explicit semantic description of each data field, where the standard is known and accepted by all parties in the communication network, in other words by using an Extensible Mark-Up language (XML).

In a preferred embodiment, the transferred data are encrypted. These encrypted data are transported with the IPsec standard.

Preferably, an authorisation establishes different access levels to the collected phenotypic and/or epigenetic parameters. The data can be accessed (decrypted) only by people with the given rights.

In the fifth step, the phenotypic data are correlated with single or multiple genetic or epigenetic parameters.

Preferably, the correlation of the epigenetic with the phenotypic parameters is done substantially without human intervention. Machine learning algorithms automatically analyse experimental data, discover systematic structure in it, and distinguish relevant parameters from uninformative ones.

In a preferred embodiment, interdependencies between two or more epigenetic parameters are taken into account.

Preferably, the revealed correlations between the phenotypic and epigenetic parameters are probabilistic in nature.

In a preferred embodiment, the formulation of the revealed relations between phenotypic and epigenetic parameters is a rule for predicting the values of selected phenotypic parameters from epigenetic parameters.

Preferably, the rule for prediction provides two or more alternative values and/or sets of values of certain phenotypic parameters with a certainty label attached to them where the sum of these certainty labels is 1.

Preferably, the formulation of the revealed relations between phenotypic and epigenetic parameters is a rule for predicting the values of selected epigenetic parameters from known phenotypic parameters.

In a preferred embodiment, the rule for prediction provides two or more alternative values and/or sets of values of selected epigenetic parameters with a certainty label attached to them where the sum of these certainty labels is 1.

Preferably, the formulation of the revealed relations between phenotypic and epigenetic parameters is a grouping of phenotypic and/or epigenetic parameters according to their relation to epigenetic parameters.

In another system according to the invention, the formulation of the revealed relations between phenotypic and epigenetic parameters is a description of a causal relationship between any two or a plurality of phenotypic and epigenetic parameters.

In a preferred embodiment, the revealed relations between phenotypic and epigenetic parameters are used for generating guidelines for investigating yet unresolved relations between any two or a plurality of epigenetic and phenotypic parameters.

In the sixth step, the revealed correlations between the phenotypic and epigenetic parameters are organised in a systematic and standardised way.

In a preferred embodiment, the epigenetic information system comprises the establishment of methods for diagnosis based on the revealed correlation between the genetic and epigenetic information and the phenotypic parameters.

Further, in another preferred embodiment the data storage, data exchange, data interpretation components are organised together within the CORBA framework.

In the seventh step, the revealed correlations between the phenotypic and epigenetic parameters are stored in a systematic and standardised way. The interpreted information integrated from different sources are amendable for storage in one unified framework.

The statistically analysed data are sent to a medical research expert device associated with pharmaceutical research experts. Preferably, the epigenetic information system comprises a suggestion of guidelines for pharmaceutical development.

In still another embodiment, the invention provides databases comprising profile data for one or more diseases which may be used in any of the above embodiments of the invention.

In another aspect of the invention, a method for treatment of an individual with a disease or medical condition is provided which comprises: (a) isolating a DNA-containing sample from an individual in need of a treatment; (b) analysing cytosine methylation patterns at selected sites of the DNA contained in the sample; (c) providing data about the methylation status at selected sites of the DNA of the individual, whereby some or all of the above-mentioned steps are carried out.

The invention further provides a computer program product for an epigenetic information system method, said computer program product comprising a computer useable storage medium having computer readable program code means embodied in the medium. The computer readable program code means include: (A) computer readable program code means for collecting and storing information about a plurality of different phenotypic parameters of collected and stored tissue samples and/or cell lines; (B) computer readable program code means for measuring and analysing multiple genetic and/or epigenetic parameters from said tissue samples and/or cell lines; (C) computer readable program code means for shipping said collected phenotypic parameters to a central or multiple distributed database in a systematic and standardised way; (D) computer readable program code means for shipping said measured genetic and/or epigenetic parameters to a central or multiple distributed database in a systematic and standardised way; (E) computer readable program code means for correlating the said phenotypic parameters with said single or multiple genetic or epigenetic parameters; (F) computer readable program code means for organising the revealed correlations in a systematic and standardised way; and (G) computer readable program code means for systematically storing said organised correlations between the phenotypic and epigenetic parameters. Using the computer readable program code means on a suited system (e.g. as mentioned below) allows performing the method of the present invention. The construction and configuration of a suited network or computer system is described in the above-indicated state of the art.

In a preferred embodiment, the computer program product for an epigenetic information system method according to the present invention further comprises computer readable program code means which contain suggestions of guidelines for pharmaceutical development. The product can further comprise computer readable program code means for establishing methods for diagnosis based on the revealed correlation between the genetic and epigenetic information and the phenotypic parameters.

In another preferred embodiment of the computer program product for an epigenetic information system method according to the invention, the phenotypic parameters contain information about an individual which includes gender and/or age and/or diagnosis and/or disease history and/or drug resistance and/or life style and/or populational information. The phenotypic parameters can contain information which are on the cellular or molecular level, whilst the epigenetic parameter can contain information about DNA methylation.

In still another embodiment, the invention provides a computer program product for an epigenetic information system method which further includes computer readable program code means for retrieving the genetic or epigenetic and phenotypic parameters from data storage computer systems that are connected to the Internet or to a like distributed data exchange system. A preferred computer program product according to the invention allows a data description and data transfer that is done by using a standard explicit semantic description of each data field, where the standard is known and accepted by all parties in the communication network, in other words by using an Extensible Mark-Up language (XML).

Furthermore, it is particularly preferred that the computer program product for an epigenetic information system method according to the invention includes computer readable program code means for encrypting the transferred data. These means are provided in order to protect the privacy and confidentiality of the patient data tat is generated and analysed using the computer program product of the present invention. Thus, additional means can be provided, that allow an authorisation to establish different access levels to the collected phenotypic and/or epigenetic parameters.

In another embodiment of the computer program product for an epigenetic information system method according to the invention, the data storage, data exchange, data interpretation components are organised together within the CORBA framework.

In order to provide an efficient and reliable working of the system according to the present invention, preferred is a computer program product for an epigenetic information system method, wherein the correlation of the epigenetic with the phenotypic parameters is done substantially without human intervention.

Further, in another preferred embodiment the computer program product of the present invention further contains computer readable program code means for taking into account the correlation interdependecies between two or more epigenetic parameters. In addition, the inventive computer program product allows revealing correlations between phenotypic and epigenetic parameters that are probabilistic in nature.

Preferred is a computer program product for an epigenetic information system method according to the present invention, wherein the formulation of the revealed relations between phenotypic and epigenetic parameters is a rule for predicting the values of selected phenotypic parameters from epigenetic parameters. Even more preferred is a product, wherein the rule for prediction provides two or more alternative values and/or sets of values of certain phenotypic parameters with a certainty label attached to them where the sum of these certainty labels is 1.

Alternatively, provided is a computer program product for an epigenetic information system method according to the present invention, wherein the formulation of the revealed relations between phenotypic and epigenetic parameters is a rule for predicting the values of selected epigenetic parameters from known phenotypic parameters. In this aspect, even more preferred is a product, wherein the rule for prediction provides two or more alternative values and/or sets of values of selected epigenetic parameters with a certainty label attached to them where the sum of these certainty labels is 1.

Another computer program product for an epigenetic information system method according to invention is characterized in that the formulation of the revealed relations between phenotypic and epigenetic parameters is a grouping of phenotypic parameters according to their relation to epigenetic parameters. In another embodiment of the computer program product according to the invention, the formulation of the revealed relations between phenotypic and epigenetic parameters is a grouping of epigenetic parameters according to their relation to epigenetic parameters.

The invention further provides a computer program product for an epigenetic information system method, wherein the formulation of the revealed relations between phenotypic and epigenetic parameters is a description of a causal relationship between any two or a plurality of phenotypic and epigenetic parameters. Preferred is a inventive computer program product which further includes computer readable program code means for using the revealed relations between phenotypic and epigenetic parameters for generating guidelines for investigating yet unresolved relations between any two or a plurality of epigenetic and phenotypic parameters.

In another aspect of the present invention, the invention provides an epigenetic information system that includes a) means for systematically collecting and storing tissue samples and/or cell lines and the phenotypic parameters about said samples; b) molecular biological system means for measuring and analysing multiple genetic and/or epigenetic parameters from said tissue samples and/or cell lines; c) means for systematically and standardised shipping of said collected phenotypic parameters to a central or multiple distributed database; d) means for systematically and standardised shipping of said measured genetic and/or epigenetic parameters to a central or multiple distributed database; e) means for correlating the said phenotypic parameters with said single or multiple genetic or epigenetic parameters; f) means for organising the revealed correlations in a systematic and standardised way; and g) means for systematically storing said organised correlations between the phenotypic and epigenetic parameters. Thus, in a complete system of the present invention, computerized means, like robots as well as mechanical means like heaters, cooling elements and shelves can be combined in order to perform the method of the present invention. The way of constructing, designing and assembly of such components of the device, including its potential connection to computer networks are common technical knowledge to the person skilled in the art. The assembly can include machines for the preparation of DNA from samples, robots for transferring the DNA thus prepared to other compartments of the system, machines for performing bisulfite reactions, PCR, and scanners, biochips, mass spectrometers, fluorescence readers and computers for the analyses of the data thus generated. Finally, means can be provided for the shipping, storing and handling of the data.

Preferred is an epigenetic information system which further includes means for suggesting guidelines for pharmaceutical development. These guidelines can be stored in a database and used to influence the decisions of the inventive system. Even more preferred is an epigenetic information system of the present invention which includes means for the establishment of methods for diagnosis based on the revealed correlation between the genetic and epigenetic information and the phenotypic parameters. According to the present invention, the epigenetic information system might further include means for describing the phenotypic parameters of an individual where the description includes gender and/or age and/or diagnosis and/or disease history and/or drug resistance and/or life style and/or populational information. This information can also be stored in a database and can be employed in order to influence the decisions of the inventive system.

In another embodiment of epigenetic information system according to the invention the phenotypic parameters are on the cellular level or on the molecular level. Furthermore, the epigenetic parameter can be DNA methylation.

In another embodiment of the present invention, the epigenetic information system further includes means for retrieving the genetic or epigenetic and phenotypic parameters from data storage computer systems that are connected to the Internet or to a like distributed data exchange system and/or means for doing the data description and data transfer by using a standard explicit semantic description of each data field, where the standard is known and accepted by all parties in the communication network, in other words by using an Extensible Mark-Up language (XML). Furthermore, the system can include means for organizing the data storage, data exchange, data interpretation components together within the CORBA framework. The use of this language and framework allows an easier exchange and handling of the retrieved data.

In another preferred embodiment of the epigenetic information system according to the invention, the system includes means for encrypting the transferred data. These means are provided in order to protect the privacy and confidentiality of the patient data tat is generated and analysed using the computer program product of the present invention. Thus, additional means can be provided, that allow an authorisation to establish different access levels to the collected phenotypic and/or epigenetic parameters.

A general aspect of the system of the present invention is related to the automatisation and handling of the material and information that is used with respect to the present invention. A general goal of the present invention is the minimisation of human interference when performing the present invention. Thus, in one aspect of the present invention, an epigenetic information system is provided, wherein the correlation of the epigenetic with the phenotypic parameters is done substantially without human intervention.

Preferably, the correlation takes into account interdependecies between two or more epigenetic parameters. Even more preferably, the revealed correlations between phenotypic and epigenetic parameters are probabilistic in nature.

In another embodiment of the epigenetic information system of the present invention, the formulation of the revealed relations between phenotypic and epigenetic parameters is a rule for predicting the values of selected phenotypic parameters from epigenetic parameters. More preferred, the rule for prediction provides two or more alternative values and/or sets of values of certain phenotypic parameters with a certainty label attached to them where the sum of these certainty labels is 1.

In still another embodiment of the epigenetic information system of the present invention, the formulation of the revealed relations between phenotypic and epigenetic parameters is a rule for predicting the values of selected epigenetic parameters from known phenotypic parameters. More preferred, the rule for prediction provides two or more alternative values and/or sets of values of selected epigenetic parameters with a certainty label attached to them where the sum of these certainty labels is 1.

In another system according to the invention, the formulation of the revealed relations between phenotypic and epigenetic parameters is a grouping of phenotypic parameters according to their relation to epigenetic parameters. In even another system according to the invention, the formulation of the revealed relations between phenotypic and epigenetic parameters is a grouping of epigenetic parameters according to their relation to epigenetic parameters.

In a preferred embodiment of the epigenetic information system of the present invention, the formulation of the revealed relations between phenotypic and epigenetic parameters is a description of a causal relationship between any two or a plurality of phenotypic and epigenetic parameters. In even another embodiment according to the invention, the revealed relations between phenotypic and epigenetic parameters are used for generating guidelines for investigating yet unresolved relations between any two or a plurality of epigenetic and phenotypic parameters.

In a final aspect of the present invention, an epigenetic information system for the treatment of an individual with a disease or medical condition is provided, which includes (a) means for isolating a DNA-containing sample from an individual; (b) means for analysing cytosine methylation patterns at selected sites of the DNA contained in the sample; and (c) means for providing data about the methylation status at selected sites of the DNA of the individual, thereby carrying out the steps of the inventive method as described above.

Alternative systems and methods for implementing the analytic methods of this invention will be apparent to one of skill in the art and are intended to be comprehended within the accompanying claims. In particular, the accompanying claims are intended to include the alternative program structures for implementing the methods of this invention that will be readily apparent to one of skill in the art.

### EXAMPLE 1

A sample to be analysed is taken from a patient and the DNA of the patient is analysed in order to obtain patient data with respect to the methylation status at selected sites of the DNA of the patient. This information is then provided to a computing device. The patient may be further examined to obtain further patient information that may include one or more of gender, age, diagnosis, disease history, drug resistance, life style and populational information and information for drug treatments or other conditions. The information may include historical information on prior therapeutic treatment regimens for the disease or medical condition. The patient information is stored in the computing device, or transferred to the computing device from another computing device, storage device or hard copy, when the information has been previously determined.

### EXAMPLE 2

Transmission of personal records: name and age of different people

First a DTD file is set up for that purpose:

personalrecords.dtd:

<name>: string; the name of the personal

<age>: integer; the age of the personal

data 1.xml:

<use dtd personalrecords.dtd>

<name> Smith

<age> 12

<name> Sholz

<age> 34

### EXAMPLE 3

This epigenetic information method comprises the steps as follows: A tissue sample from a patient suffering from an insufficiently specified acute disease is taken by medical personnel in a medical setting. In the context of the present invention, the term “insufficiently specified acute disease” designates a generally diagnosed disease like, for example, cancer without specifying the exact type of cancer the patient is affected with. Basically all types of samples that contain DNA from the patient can be employed in the method of the present invention. The sample can contain either specific tissue cells, like single types of blood cells, single types of liver cells or cells of a single tumour, or more generally any kind of tissue e.g. skin, brain or other organs.

The sample is then shipped together with additional patient information to a central laboratory in order to analyse the methylation states with a kit at selected sites of the patients' DNA.

The base cytosine, but not 5-methylcytosine, from the thus obtained genomic DNA is then converted into uracil by treatment with a bisulfite solution.

Fractions of the thus chemically treated genomic DNA are amplified using the polymerase chain reaction. Different methylation states of individual CpG dinucleotides are then determined using hybridisation probes that are specific for that particular CpG. Since the hybridisation probes are used as a microarray comprising many different probes, many different CpGs can be analysed simultanously. Reading the hybridisation signals with commercial instruments the data generated thereby are automatically applied to a processing algorithm. This allows the drawing of conclusions concerning the phenotype of the sample material. Collected phenotypic parameters are shipped in a systematic and standardised way to the central database located in a hospital. The measured genetic parameters and cytosine methylation patterns are sent to the same server of the hospital. There the phenotypic data are correlated with single or multiple genetic parameters and cytosine methylation patterns. Listings of precisely defined diseases of the individual patient are generated and stored in a systematic and standardised way.

### EXAMPLE 4

Tissue Collection and Storage.

Tissue and cell line samples may be collected from multiple sources, examples of sources include hospitals, reference laboratories and universities. Each tissue sample or cell line sample is given a sample identifier (sample ID). Upon receipt, each sample is designated a unique identifier code (reference ID) comprising of an identifier designating the source of the tissue and the sample ID. Patient and sample data are collected from the source under the same sample ID, and therefore subsequently given the same reference ID. If the sample is a follow-up sample, i.e. this is not the first sample for one individual patient, this is indicated at the source upon a data sheet. Upon arrival, each sample receives a barcode, the information is entered into the internal database and the barcode is linked to the patient information.

Standardisation of Data Concerning Phenotypic Parameters of Samples.

Data concerning the phenotypic parameters of the sample (for example, diagnosis, disease progression etc..) are standardised at the source in such a manner that they are compatible to or identical to the internal database that they are subsequently stored upon.

For example, if breast cancer samples are collected, at the source of the sample a diagnosis of ductal carcinoma in situ may be identified by entering “ductal carcinoma in situ” or “DCIS” or a code, e.g. “9” (with an attached table explaining that 9=DCIS). Once received, the samples are marked according to the internal standard e.g. ‘DCIS’. Wherein the source uses another term, this can be easily mapped, by inclusion of a key allowing the translation of terminology from the standard of the source organisation to the standard to be used in the database. This also allows import of data from several sources in different formats into the internal database, as long as the source institutions follow a standardized or logical methodology of describing phenotypic parameters of a sample.

The database is designed such that it may carry details of multiple samples originating from a single patient. Parameters to be included in the database may include the following:

indication from which anatomical location the tissue was taken

indication of the tissue was considered sick or healthy macroscopically/microscopically

indication of when in the disease course the sample was taken (e.g. before chemotherapy or during/after)

diagnosis and therapy and follow-up data (e.g. disease-free and overall survival) regarding the patient and the linkage of samples to these data

Technical information regarding the samples may also be recorded, e.g. shipping conditions, location, amount etc.

It may be required to monitor progression regarding some of the fields, e.g. survival times (e.g. disease-free survival<overall survival), diagnosis (e.g. prostate cancer, only in male patients).

Measurement and Analysis of Genetic and/or Epigenetic Parameters.

In the first step the genomic DNA is isolated from the cell samples using the Wizzard kit from (Promega) and digested with MssI (MBI Fermentas, St. Leon-Rot, Germany).

The isolated genomic DNA from the samples is treated using a bisulfite solution (hydrogen sulfite, disulfite). The treatment is such that all non methylated cytosines within the sample are converted to thymidine, conversely 5-methylated cytosines within the sample remain unmodified. Bisulphite treatment of genomic DNA was done with minor modifications as described by A. Olek, J. Oswald, J. Walter, Nucleic Acid Res. 24, 5064 (1996). prior to the modification by bisulphite.

The treated nucleic acids are then amplified using multiplex PCRs, amplifying 8 fragments per reaction with Cy5 fluorescently labelled primers. Primers design is carried out according to the guidelines of Clark and Frommer (S. J. Clark, M. Frommer, in Laboratory Methods for the Detection of Mutations and Polymorphisms in DNA, G. R. Taylor ed. (CRC Press, Boca Raton 1997)).

10 ng DNA is used as template DNA for each PCR reactions. The template DNA, 12.5 pmol or 40 pmol (CY5-labelled) of each primer, 0.5-2 U Taq polymerase (HotStarTaq, Qiagen, Hilden, Germany) and 1 mM dNTPs are incubated with the reaction buffer supplied with the enzyme in a total volume of 20 μl. After activation of the enzyme (15 min, 96° C.) the incubation times and temperatures are 95° C. for 1 min followed by 34 cycles (95° C. for 1 min, annealing temperature (see Supplementary information) for 45 sec, 72° C. for 75 sec) and 72° C. for 10 min.

All PCR products from each individual sample are then hybridised to glass slides carrying a pair of immobilised oligonucleotides for each CpG position under analysis. Each of these detection oligonucleotides are designed to hybridise to the bisulphite converted sequence around one CpG site which was either originally unmethylated (TG) or methylated (CG). Hybridisation conditions are selected to allow the detection of the single nucleotide differences between the TG and CG variants.

Oligonucleotides with a C6-amino modification at the 5′end are spotted with 4-fold redundancy on activated glass slides (T. R. Golub et al., Science 286, 531 (1999)). For each analysed CpG position two oligonucleotides N₍₂₋₁₆₎-CG-N₍₂₋₁₆₎ and N₍₂₋₆₎-TG-N₍₂₋₁₆₎, reflecting the methylated and non methylated status of the CpG dinucleotides, were spotted and immobilised on the glass array. Subsequently, the fluorescent images of the hybridised slides are visualised using a GenePix 4000 microarray scanner (Axon Instruments).

Shipping of Phenotypic Parameters to Database or Databases.

The description of the biological sample (e.g. phenotypic characteristics, methylation profile) is entered into a preformated questionnaire electronically. The questionnaire contains also all possible values for all fields, that are predefined and should be consistent with previous entries of the databases containing sample information. If data integrity is of a great importance double/triple data entry is performed. The data entry forms may be extended or modified on a case by case basis if necessary, however they should essentially be standardised and should be identical between different studies.

The entered data is then parsed by computer software—and entered into the database that organises the sample information concerning a specific study. If possible, consistency checks are performed. The rules for the consistency check are predefined on a case by case basis.

Sample descriptions are linked with physical samples via a reference id or sample ID that are given as described above. The reference id or sample ID are therefore present on the physical sample as well as in the table that contains the description for the sample.

Shipping of measured phenotypic parameters to database or databases. The flow of the measurement values can be summarised as follows:

1. Laboratory management system (LIMS): workflow management

2. LIMS database: experimental tracking

3. Raw data database: storing the raw data as the direct output of the measurment units

4. Data warehouse: storage of the raw measurment values integrated with all important genetic, sample related and experimental parameters for the purpose of data interpretation.

The experimental workflows are managed and tracked by a laboratory management system (LIMS). The LIMS records all essential parameters and work steps for each measurement or series of measurements. These recorded paramteres are then stored in the database for the LIMS. These parameteres include reagents, measured genomic locations, measured samples, dates of measurements, etc.

The result of each measurement is then recorded in a digitised form and then organised into a database with unique references.

Taking a microarray study as an example, the unique reference for the measurement is the ID that is assigned during the grid alignment. This ID is unique in the whole system. This ID allows the identification of the experimental step of array scanning as well as the barcode of the microarray. Via the microarray barcode all further parameters can be identified such as the chip layout, the composition of the probe, etc. The database table for storing raw data contains the following fields: grid alignment ID; oligonuclotide ID; oligonuclotide coordinate; oligonuclotide sequence; foreground intensity; background intensity; pixel standard deviation.

In the next step all information sets are integrated together:

1. sample description obtained previously

2. experimental paramteres recorded by the LIMS

3. raw measurement values.

This integration of different data sources takes place in a data warehouse, a database that is organised for the purpose of data interpretation. The integration is achieved by using the above-mentioned unique keys for measurement values and the corresponding unique identifier for the biological sample that was used in the experiment.

All of these above described software components are integrated together in a CORBA framework. This software architecture allows the distribution of different components onto different computer platforms and allows different computers to communicate via a network. The interpretation of the raw measurement values is then obtained by submitting queries in the data warehouse (DWH).

Correlation of Phenotypic Parameters with Genetic or Epigenetic Parameters

First the data is obtained by submitting queries into the DWH. For example, all microarray methylation data is requested that belong to a certain project and originate from samples from a certain origin, e.g. prostate biopsy. These data sets are organised into two groups a diseased (e.g. prostate carcinoma samples) and the control group (e.g. samples with benign prostate hyperplasia, hereinafter referred to as BPH).

Individual methylation sites, CpGs, that were measured on the obtained microarrays are then ranked according to their informativeness with respect to distinguishing prostate cancer samples from the BPH samples. Several methods can be used for this ranking step, described elsewhere (F. Model, P. Adorjan, A. Olek and C. Piepenbrock, “Feature selection for DNA methylation based cancer classification”, Bioinformatics, 17 Suppl 1, S157-64, 2001).

Organisation of Correlations in a Systematic and Standardised Manner

The methylation patterns of those CpGs that are identified in the previous step as informative are then represented in ranked matrixes, where each line represents an individual CpG position, each column an individual patient, and the colour of each block represents the methylation level of the particular CpG at the particular sample.

Steps e) and f) according to Claim 1 are achieved by using a proprietary software developed for this particular purpose, called “Mana” (methylation analyzer). A more standardised and routine organisation of the revealed correlations can be done by using a mark up language, such as XML. The proprietary software tool, “StarGEM” parses an XML software code that describes all biological sample classes to be compared as well as all methods for correlating methylation patterns with the phenotypic parameters. Results are then automatically organised into a report in HTML or PDF format. This report is generated automatically, therefore it has a standardised layout.

Systematic Organisation of Correlations Between the Phenotypic and Epigenetic Parameters.

This representation contains two components:

1. The revealed genetic/epigenetic patterns in relation to the investigated samples.

2. The method of obtaining said correlations between the phenotypic and epigenetic parameteres.

The revealed genetic/epigenetic patterns should directly represent the genetic/epigenetic parameter of interest. For example a number is stored that is in direct correlation with the methylation level at a measured CpG site. The number may be furthermore calibrated such that it expresses the methylation level in methylation percent. The data is linked in a relational database structure to the sample description that has been obtained previously. Furthermore it is linked to a genetic location in the genetic map of the investigated organism. For example it relates all measured CpG sites to accession numbers for the DNA sequence revealed by the human genome project. This makes it possible to query complex genetic/epigenetic correlations with phenotypic parameters. For example one can submit a query to obtain all genetic locations that are differentially methylated between prostate carcinoma tissue samples and BPH samples.

The method of obtaining said correlations between the phenotypic and epigenetic parameters could be highly complex, and it may change between different experiments or between different platforms. For example different data manipulation procedure should be used for preprocessing mRNA microarray data than preprocessing methylation microarray data. It follows that an exact representation of the data manipulation procedure is required. This is achieved by describing the complete workspace for the data interpretation in an XML structure. The software that is used to reveal said correlations is able to write and parse such an XML description of the complete data interpretation flow. This means that the complete data interpretation flow can be obtained and visualised upon request. This is essential to reproduce all revealed correlations between genetic/epigenetic parameters and phenotypic parameters at any time.

What is claimed is:

1. An epigenetic information system method comprising
a) a systematic way of collecting and storing
tissue samples and/or cell lines
the phenotypic parameters about said samples
b) a molecular biological system for measuring and analysing multiple genetic and/or epgenetic parameters from said tissue samples and/or cell lines;
c) a systematic and standardised way of shipping said collected phenotypic parameters to a central or multiple distributed database;
d) a systematic and standardised way of shipping said measured genetic and/or epigenetic parameters to a central or multiple distributed database;
e) means for correlating the said phenotypic parameters with said single or multiple genetic or epigenetic parameters;
f) means for organising the revealed correlations in a systematic and standardised way; and
g) a systematic way of storing said organised correlations between the phenotypic and epigenetic parameters.

2. A method according to claim (1) where the epigenetic information system comprises a suggestion of guidelines for pharmaceutical development.

3. A method according to claim (1) where the epigenetic information system comprises the establishment of methods for diagnosis based on the revealed correlation between the genetic and epigenetic information and the phenotypic parameters.

4. A method according to claim (1) where the phenotypic parameters describe an individual where the description includes gender and/or age and/or diagnosis and/or disease history and/or drug resistance and/or life style and/or populational information.

5. A method according to claim (1) where the phenotypic parameters are on the cellular level.

6. A method according to claim (1) where the phenotypic parameters are on the molecular level.

7. A method according to claim (1) where the epigenetic parameter is DNA methylation.

8. A method according to claim (1) where the genetic or epigenetic and phenotypic parameters are retrieved from data storage computer systems that are connected to the Internet or to a like distributed data exchange system.

9. A method according to claim (8) where the data description and data transfer is done by using a standard explicit semantic description of each data field, where the standard is known and accepted by all parties in the communication network, in other words by using an Extensible Mark-Up language (XML).

10. A method according to claim (9) where the transferred data are encrypted.

11. A method according to claim (1) where an authorisation establishes different access levels to the collected phenotypic and/or epigenetic parameters.

12. A method according to claim (1) where the data storage, data exchange, data interpretation components are organised together within the CORBA framework.

13. A method according to claim (1) where the correlation of the epigenetic with the phenotypic parameters is done substantially without human intervention.

14. A method according to claim (1) where the correlation takes into account interdependecies between two or more epigenetic parameters.

15. A method according to claim (1) where the revealed correlations between phenotypic and epigenetic parameters are probabilistic in nature.

16. A method according to claim (1) where the formulation of the revealed relations between phenotypic and epigenetic parameters is a rule for predicting the values of selected phenotypic parameters from epigenetic parameters.

17. A method according to claim (16) where the rule for prediction provides two or more alternative values and/or sets of values of certain phenotypic parameters with a certainty label attached to them where the sum of these certainty labels is 1.

18. A method according to claim (1) where the formulation of the revealed relations between phenotypic and epigenetic parameters is a rule for predicting the values of selected epigenetic parameters from known phenotypic parameters.

19. A method according to claim (18) where the rule for prediction provides two or more alternative values and/or sets of values of selected epigenetic parameters with a certainty label attached to them where the sum of these certainty labels is 1.

20. A method according to claim (1) where the formulation of the revealed relations between phenotypic and epigenetic parameters is a grouping of phenotypic parameters according to their relation to epigenetic parameters.

21. A method according to claim (1) where the formulation of the revealed relations between phenotypic and epigenetic parameters is a grouping of epigenetic parameters according to their relation to epigenetic parameters.

22. A method according to claim (1) where the formulation of the revealed relations between phenotypic and epigenetic parameters is a description of a causal relationship between any two or a plurality of phenotypic and epigenetic parameters.

23. A method according to claim (1) where the revealed relations between phenotypic and epigenetic parameters are used for generating guidelines for investigating yet unresolved relations between any two or a plurality of epigenetic and phenotypic parameters.

24. A method for treatment of an individual with a disease or medical condition, the method comprising:
(a) isolating a DNA-containing sample from an individual;
(b) analysing cytosine methylation patterns at selected sites of the DNA contained in the sample;
(c) providing data about the methylation status at selected sites of the DNA of the individual, thereby carrying out the method of claim 1.

25. A computer program product for an epigenetic information system method, said computer program product comprising a computer useable storage medium having computer readable program code means embodied in the medium, the computer readable program code means comprising:
(A) computer readable program code means for collecting and storing information about a plurality of different phenotypic parameters of collected and stored tissue samples and/or cell lines;
(B) computer readable program code means for measuring and analysing multiple genetic and/or epigenetic parameters from said tissue samples and/or cell lines;
(C) computer readable program code means for shipping said collected phenotypic parameters to a central or multiple distributed database in a systematic and standardised way;
(D) computer readable program code means for shipping said measured genetic and/or epigenetic parameters to a central or multiple distributed database in a systematic and standardised way;
(E) computer readable program code means for correlating the said phenotypic parameters with said single or multiple genetic or epigenetic parameters;
(F) computer readable program code means for organising the revealed correlations in a systematic and standardised way; and
(G) computer readable program code means for systematically storing said organised correlations between the phenotypic and epigenetic parameters.

26. A computer program product for an epigenetic information system method according to claim 25, said product further comprising:
(H) computer readable program code means comprising suggestions of guidelines for pharmaceutical development.

27. A computer program product for an epigenetic information system method according to claim 25, said product further comprising:
(I) computer readable program code means for establishing methods for diagnosis based on the revealed correlation between the genetic and epigenetic information and the phenotypic parameters.

28. A computer program product for an epigenetic information system method according to claim 25, wherein the phenotypic parameters contain information about an individual which includes gender and/or age and/or diagnosis and/or disease history and/or drug resistance and/or life style and/or populational information.

29. A computer program product for an epigenetic information system method according to claim 25, wherein the phenotypic parameters contain information which are on the cellular or molecular level.

30. A computer program product for an epigenetic information system method according to claim 25, wherein the epigenetic parameter contains information about DNA methylation.

31. A computer program product for an epigenetic information system method according to claim 25 said product further comprising:
(J) computer readable program code means for retreiving the genetic or epigenetic and phenotypic parameters from data storage computer systems that are connected to the Internet or to a like distributed data exchange system.

32. A computer program product for an epigenetic information system method according to claim 25, allowing a data description and data transfer that is done by using a standard explicit semantic description of each data field, where the standard is known and accepted by all parties in the communication network, in other words by using an Extensible Mark-Up language (XML).

33. A computer program product for an epigenetic information system method according to claim 25, said product further comprising:
(K) computer readable program code means for encrypting the transferred data.

34. A computer program product for an epigenetic information system method according to claim 25, wherein the product allows an authorisation to establish different access levels to the collected phenotypic and/or epigenetic parameters.

35. A computer program product for an epigenetic information system method according to claim 25, wherein the data storage, data exchange, data interpretation components are organised together within the CORBA framework.

36. A computer program product for an epigenetic information system method according to claim 25, wherein the correlation of the epigenetic with the phenotypic parameters is done substantially without human intervention.

37. A computer program product for an epigenetic information system method according to claim 25, said product further comprising:
(K) computer readable program code means for taking into account the correlation interdependecies between two or more epigenetic parameters.

38. A computer program product for an epigenetic information system method according to claim 25, that allows revealing correlations between phenotypic and epigenetic parameters that are probabilistic in nature.

39. A computer program product for an epigenetic information system method according to claim 25, wherein the formulation of the revealed relations between phenotypic and epigenetic parameters is a rule for predicting the values of selected phenotypic parameters from epigenetic parameters.

40. A computer program product for an epigenetic information system method according to claim 39, wherein the rule for prediction provides two or more alternative values and/or sets of values of certain phenotypic parameters with a certainty label attached to them where the sum of these certainty labels is 1.

41. A computer program product for an epigenetic information system method according to claim 25, wherein the formulation of the revealed relations between phenotypic and epigenetic parameters is a rule for predicting the values of selected epigenetic parameters from known phenotypic parameters.

42. A computer program product for an epigenetic information system method according to claim 41, wherein the rule for prediction provides two or more alternative values and/or sets of values of selected epigenetic parameters with a certainty label attached to them where the sum of these certainty labels is 1.

43. A computer program product for an epigenetic information system method according to claim 39, wherein the formulation of the revealed relations between phenotypic and epigenetic parameters is a grouping of phenotypic parameters according to their relation to epigenetic parameters.

44. A computer program product for an epigenetic information system method according to claim 39, wherein the formulation of the revealed relations between phenotypic and epigenetic parameters is a grouping of epigenetic parameters according to their relation to epigenetic parameters.

45. A computer program product for an epigenetic information system method according to claim 39, wherein the formulation of the revealed relations between phenotypic and epigenetic parameters is a description of a causal relationship between any two or a plurality of phenotypic and epigenetic parameters.

46. A computer program product for an epigenetic information system method according to claim 25, said product further comprising:
(K) computer readable program code means for using the revealed relations between phenotypic and epigenetic parameters for generating guidelines for investigating yet unresolved relations between any two or a plurality of epigenetic and phenotypic parameters.

47. An epigenetic information system comprising:
a) means for systematically collecting and storing
tissue samples and/or cell lines
the phenotypic parameters about said samples
b) molecular biological system means for measuring and analysing multiple genetic and/or epigenetic parameters from said tissue samples and/or cell lines;
c) means for systematically and standardised shipping of said collected phenotypic parameters to a central or multiple distributed database;
d) means for systematically and standardised shipping of said measured genetic and/or epigenetic parameters to a central or multiple distributed database;
e) means for correlating the said phenotypic parameters with said single or multiple genetic or epigenetic parameters;
f) means for organising the revealed correlations in a systematic and standardised way; and
g) means for systematically storing said organised correlations between the phenotypic and epigenetic parameters.

48. The epigenetic information system of claim (47), further comprising:
h) means for suggesting guidelines for pharmaceutical development.

49. The epigenetic information system of claim (47), further comprising:
h) means for the establishment of methods for diagnosis based on the revealed correlation between the genetic and epigenetic information and the phenotypic parameters.

50. The epigenetic information system of claim (47), further comprising:
i) means for describing the phenotypic parameters of an individual where the description includes gender and/or age and/or diagnosis and/or disease history and/or drug resistance and/or life style and/or populational information.

51. The epigenetic information system of claim (47), where the phenotypic parameters are on the cellular level.

52. The epigenetic information system of claim (47), where the phenotypic parameters are on the molecular level.

53. The epigenetic information system of claim (47), where the epigenetic parameter is DNA methylation.

54. The epigenetic information system of claim (47), further comprising:
j) means for retrieving the genetic or epigenetic and phenotypic parameters from data storage computer systems that are connected to the Internet or to a like distributed data exchange system.

55. The epigenetic information system of claim (47), further comprising:
k) means for doing the data description and data transfer by using a standard explicit semantic description of each data field, where the standard is known and accepted by all parties in the communication network, in other words by using an Extensible Mark-Up language (XML).

56. The epigenetic information system of claim (47), further comprising:
l) means for encrypting the transferred data.

57. The epigenetic information system of claim (47), wherein an authorisation establishes different access levels to the collected phenotypic and/or epigenetic parameters.

58. The epigenetic information system of claim (47), further comprising:
m) means for organizing the data storage, data exchange, data interpretation components together within the CORBA framework.

59. The epigenetic information system of claim (47), wherein the correlation of the epigenetic with the phenotypic parameters is done substantially without human intervention.

60. The epigenetic information system of claim (47), wherein the correlation takes into account interdependecies between two or more epigenetic parameters.

61. The epigenetic information system of claim (47), wherein the revealed correlations between phenotypic and epigenetic parameters are probabilistic in nature.

62. The epigenetic information system of claim (47), wherein the formulation of the revealed relations between phenotypic and epigenetic parameters is a rule for predicting the values of selected phenotypic parameters from epigenetic parameters.

63. The epigenetic information system of claim (62), wherein the rule for prediction provides two or more alternative values and/or sets of values of certain phenotypic parameters with a certainty label attached to them where the sum of these certainty labels is 1.

64. The epigenetic information system of claim (47), wherein the formulation of the revealed relations between phenotypic and epigenetic parameters is a rule for predicting the values of selected epigenetic parameters from known phenotypic parameters.

65. The epigenetic information system of claim (64), wherein the rule for prediction provides two or more alternative values and/or sets of values of selected epigenetic parameters with a certainty label attached to them where the sum of these certainty labels is 1.

66. The epigenetic information system of claim (47), wherein the formulation of the revealed relations between phenotypic and epigenetic parameters is a grouping of phenotypic parameters according to their relation to epigenetic parameters.

67. The epigenetic information system of claim (47), wherein the formulation of the revealed relations between phenotypic and epigenetic parameters is a grouping of epigenetic parameters according to their relation to epigenetic parameters.

68. The epigenetic information system of claim (47), wherein the formulation of the revealed relations between phenotypic and epigenetic parameters is a description of a causal relationship between any two or a plurality of phenotypic and epigenetic parameters.

69. The epigenetic information system of claim (47), wherein the revealed relations between phenotypic and epigenetic parameters are used for generating guidelines for investigating yet unresolved relations between any two or a plurality of epigenetic and phenotypic parameters.

70. An epigenetic information system for the treatment of an individual with a disease or medical condition, the system comprising:
(a) means for isolating a DNA-containing sample from an individual;
(b) means for analysing cytosine methylation patterns at selected sites of the DNA contained in the sample; and
(c) means for providing data about the methylation status at selected sites of the DNA of the individual, thereby carrying out the method of claim 1.
